(19) United States
(12) Patent Application Publication
(10) Pub. No.: US 2026/0174825 A1
(43) Pub. Date: Jun. 25, 2026

(54) BIOMIMETIC CHIP, METHOD FOR MANUFACTURING SAME, AND METHOD FOR COATING EXTRACELLULAR MATRIX BY USING SAME

(71) Applicant: UNIST (ULSAN NATIONAL INSTITUTE OF SCIENCE AND TECHNOLOGY), Ulsan (KR)

(72) Inventors: Joo Hun KANG, Ulsan (KR); Juhae CHOI, Ulsan (KR)

(73) Assignee: UNIST (ULSAN NATIONAL INSTITUTE OF SCIENCE AND TECHNOLOGY), Ulsan (KR)

(21) Appl. No.: 19/436,099

(22) Filed: Dec. 30, 2025

Related U.S. Application Data
(63) Continuation of application No. PCT/KR2025/099584, filed on Mar. 6, 2025.

(30) Foreign Application Priority Data
Mar. 6, 2024 (KR) 10-2024-0032124
Feb. 7, 2025 (KR) 10-2025-0016232

(51) Int. Cl. A61K 38/00; A61L 27/36

(52) U.S. Cl. CPC A61K 38/00; A61L 27/3633

(57) ABSTRACT

Provided is a microphysiological system including a substrate having a first groove, a cover part disposed opposite the substrate and having a second groove formed on a surface thereof facing the substrate, a porous membrane disposed between the substrate and the cover part so as to have an area overlapping at least the first groove and the second groove, and a plurality of openings having a first inlet, a first outlet, a second inlet, and a second outlet, which pass through the cover part in a thickness direction or in a direction perpendicular to the thickness direction. The microphysiological system maintains a constant circulation velocity of fluid within a microfluidic channel to induce uniform-density adhesion of cells on the surface of the microfluidic channel during cell culture and circulation, and is bonded by using oxygen plasma and a silane coupling agent and thus attached immediately and strongly through chemical bonding.

### CROSS-REFERENCE TO RELATED APPLICATIONS

This application is a continuation of International Application No. PCT/KR 2025/099584 filed on Mar. 6, 2025, which claims priority to Korean Patent Application No. 10-2024-0032124 filed on Mar. 6, 2024 and Korean Patent Application No. 10-2025-0016232 filed on Feb. 7, 2025, the entire contents of which are herein incorporated by reference.

### TECHNICAL FIELD

The present disclosure relates to a microphysiological system (MPS), an MPS manufacturing method, and an extracellular matrix coating method using the MPS.

The present application is the result of research conducted with the support of the National Research Foundation of Korea ((No. 00399800, CRISPR-based Cancer Metastasis Prediction Patient-Derived Organoid Multi-Organ Chip Laboratory (Ulsan National Institute of Science and Technology), Project No. 2710085555) and (No. 00344187, Development of High-Density Vascularized and Neuralized 3D Artificial Muscle Tissue Transplant (Ulsan National Institute of Science and Technology), Project No. 2710084180)), the Inter-Ministerial Regenerative Medicine Technology Development Program (No. 00070678, Development of Large-Area Skin and Lymphatic Vessel Reconstruction Technology Based on Vascularized 3D Autologous Tissue Construct (Ulsan National Institute of Science and Technology), Project No. 2710033096), and Donggrami Foundation (Development of NTM lung disease treatment technology based on microvesicle mimic technology that increases bacterial cell membrane permeability with biofilm/macrophage latent bacteria targeting function (Ulsan National Institute of Science and Technology) in 2025, with funding from the government (Ministry of Science and ICT) in 2025.

### BACKGROUND ART

A microphysiological system (MPS), which reproduces the structure and microenvironment of human organs in vitro by combining human-derived three-dimensional cell culture technology and blood flow-simulating microfluidic technology, is a technology that immobilizes cells within microfluidic channels in a certain space for a certain period of time and observes cell growth or the like.

An MPS is utilized in various fields such as disease mechanisms, new drug screening, or patient-tailored medical technologies, and microfluidic channels inside chips are applied in various designs such as single, double, and multi-channels depending on the purpose of use.

In an MPS for applying a cell circulation model, a conventional microfluidic channel has a curve structure. In case that the fluid flow is applied to the microfluidic channel having a curve structure so as to simulate circulating cells, the difference in fluid velocity may occur as the fluid flow is concentrated inside the curve. Due to the difference in fluid velocity in the curve area, a coating agent used for surface modification of the microfluidic channel accumulates in the curve area, making uniform surface treatment of the microfluidic channel difficult and causing uneven adhesion of cells cultured on the surface of the microfluidic channel during subsequent cell culture.

An extracellular matrix may be coated on the microfluidic channel so as to immobilize cells within the microfluidic channel in a certain space for a certain period of time and observe cell growth or the like.

Currently commercialized extracellular matrix coating methods are typically represented by simple coatings using single proteins, such as collagen, fibronectin, or laminin, or coatings using composite basement membrane proteins, such as Matrigel. These existing coating methods employ simple immersion or adsorption methods, and are thus limited to forming only two-dimensional structures having random orientation. Therefore, such methods fail to effectively reflect the sophisticated structural characteristics of in vivo basement membranes and have limitations in simulating in vivo complex extracellular matrix environments.

### DISCLOSURE OF INVENTION

### Technical Problem

In the present disclosure, cell culture and cell circulation models may be easily applied to a microphysiological system (MPS) through introduction of a microfluidic channel having a linear structure.

Furthermore, a robust cell barrier similar to an in vivo environment may be implemented.

### Solution to Problem

To solve the objects described above, an embodiment of the present disclosure relates to a microphysiological system including a substrate having a first groove formed on a surface thereof, a cover part disposed opposite the substrate and having a second groove formed on a surface thereof facing the substrate, a porous membrane disposed between the substrate and the cover part so as to have an area overlapping at least the first groove and the second groove, and a plurality of openings having a first inlet, a first outlet, a second inlet, and a second outlet, which pass through the cover part in a thickness direction or in a direction perpendicular to the thickness direction.

### Advantageous Effects of Invention

A microphysiological system according to an embodiment of the present disclosure may maintain a constant circulation velocity of fluid within a microfluidic channel to induce uniform-density adhesion of cells on the surface of the microfluidic channel during cell culture and circulation, and may be bonded by using oxygen plasma and a silane coupling agent and thus attached immediately and strongly through chemical bonding.

Furthermore, an extracellular matrix coating method according to an embodiment of the present disclosure may optimize cell-matrix interaction by introducing an aligned fibrous structure into a porous membrane, thereby effectively implementing a function of an in vivo basement membrane and strengthening a function of a cell barrier.

### BRIEF DESCRIPTION OF DRAWINGS

FIG. 1 is an exploded perspective view schematically illustrating an example of a microphysiological system (MPS) according to an embodiment of the present disclosure.

FIG. 2 is a cross-sectional view schematically illustrating an example of the MPS of FIG. 1.

FIG. 3 is a flowchart illustrating an example of an MPS manufacturing method according to another embodiment of the present disclosure.

FIG. 4 is a process diagram schematically illustrating an example of the MPS manufacturing method of FIG. 3.

FIG. 5 is a graph showing a bonding strength of a silane coupling agent according to a solvent.

FIG. 6 is an image showing a cell culture result of an MPS manufactured by the MPS manufacturing method of FIG. 3.

FIGS. 7A, 7B, 7C are images showing a test result of the MPS manufactured by the MPS manufacturing method of FIG. 3.

FIG. 8 is a flowchart illustrating an example of an extracellular matrix coating method according to another embodiment of the present disclosure.

FIG. 9 is a diagram schematically illustrating an example of the extracellular matrix coating method of FIG. 8.

FIGS. 10A and 10B are images showing the alignment of the extracellular matrix coated by the extracellular matrix coating method of FIG. 8.

FIG. 11 is a fluorescence analysis image of cells cultured on an extracellular matrix coated by the extracellular matrix coating method of FIG. 8.

FIGS. 12A, 12,B, 12C are graphs evaluating the functions of cells cultured on extracellular matrices coated under various conditions.

FIG. 13A is a fluorescence analysis image of a co-culture of cells cultured on extracellular matrices under various conditions and a cancer cell line.

FIG. 13B is a graph showing relative quantification of fluorescence-labeled tight junction proteins in FIG. 13A.

FIG. 14A is a fluorescence analysis image of metastasis of cancer cells when cells cultured on extracellular matrices under various conditions and a cancer cell line were co-cultured.

FIG. 14B is a graph showing the number of fluorescence-labeled cancer cells per volume in FIG. 14A.

FIG. 14C is a graph showing the permeability coefficient due to metastasis of cancer cells when cells cultured on extracellular matrices under various conditions and a cancer cell line were co-cultured.

### BEST MODE FOR CARRYING OUT THE INVENTION

To solve the objects described above, an embodiment of the present disclosure relates to a microphysiological system (MPS) including a substrate having a first groove formed on a surface thereof, a cover part disposed opposite the substrate and having a second groove formed on a surface thereof facing the substrate, a porous membrane disposed between the substrate and the cover part so as to have an area overlapping at least the first groove and the second groove, and a plurality of openings having a first inlet, a first outlet, a second inlet, and a second outlet, which pass through the cover part in a thickness direction or in a direction perpendicular to the thickness direction.

A length of the first groove may be greater than or equal to a length of the second groove, the first inlet and the first outlet may correspond to end portions of the first groove, respectively, and the second inlet and the second outlet may correspond to end portions of the second groove, respectively.

The first groove may have a length extending in a single direction and a width crossing the length, and the width may include a plurality of convex areas spaced apart from each other with a larger width along the length.

The plurality of convex areas may overlap the first inlet, the first outlet, the second inlet, and the second outlet.

A biofluid may be injected into the first groove and the second groove.

The biofluid may include a material selected from circulating cells, adherent cells, scaffolds, chemicals, and biomolecules.

The biofluid may be selectively exchanged through the porous membrane in an area where the first groove and the second groove overlap each other.

The circulating cells and the adherent cells may be cultured in a single-layer or multi-layer structure or may be cultured while circulating along the channels of the MPS.

The scaffolds may include an extracellular matrix protein or a hydrogel protein.

The chemicals may include cell culture media and additives.

The biomolecules may include signaling molecules or neurotransmitters.

To solve the objects described above, another embodiment of the present disclosure discloses a method of manufacturing a microphysiological system including preparing a substrate, a cover part, and a porous membrane, surface-treating the substrate and the cover part with plasma gas, surface-treating the porous membrane with a silane coupling agent, and bonding the surface-treated substrate, the surface-treated cover part, and the surface-treated porous membrane.

The plasma gas may include oxygen or air, and the oxygen or the air may modify surfaces of the substrate and the cover part with a hydroxyl group.

The silane coupling agent may include water or ethanol as a solvent.

To solve the objects described above, another embodiment of the present disclosure provides an extracellular matrix coating method including introducing a solution including an extracellular matrix protein onto a substrate, forming a constant flow in the solution, coating the extracellular matrix protein so as to be aligned directionally in a direction of the flow, and introducing cells onto the extracellular matrix coating having the directionality.

Blood plasma and calcium chloride (CaCl₂) may be further injected during the coating while forming the constant flow.

### Mode for the Invention

Prior to detailed descriptions of preferred embodiments of the present disclosure, the terms or words used in the present specification and the claims should not be construed as being limited to ordinary or dictionary meanings and should be construed as meanings and concepts consistent with the technical idea of the present disclosure.

As the present description allows for various changes and numerous embodiments, certain embodiments will be illustrated in the drawings and described in detail in the written description. Effects and features of the present disclosure, and methods of achieving them will be clarified with reference to one or more embodiments described below in detail with reference to the drawings. However, the present disclosure is not limited to the following embodiments and may be embodied in various forms.

It will be understood that although the terms “first,” “second,” etc. may be used herein to describe various elements, these elements should not be limited by these terms. These terms are only used to distinguish one element from another.

The singular forms as used herein are intended to include the plural forms as well unless the context clearly indicates otherwise.

It will be further understood that the terms “include” and/or “comprise” used herein specify the presence of stated features or elements, but do not preclude the presence or addition of one or more other features or elements.

It will be further understood that, when a layer, region, or element is referred to as being “on” another layer, region, or element, it may be directly or indirectly on the other layer, region, or element. That is, for example, intervening layers, regions, or elements may be present.

Identification symbols for operations are used for convenience of explanation and do not describe the order of the operations. The respective operations may be performed in an order different from the stated order unless the context clearly indicates otherwise. That is, the respective operations may be performed in the same order as the stated order, may be performed substantially simultaneously, or may be performed in an opposite order.

Hereinafter, embodiments of the present disclosure are described. However, the scope of the present disclosure is not limited to the following preferred embodiments, and various modifications may be made within the scope of the present disclosure by those of ordinary skill in the art.

Also, sizes of elements in the drawings may be exaggerated or reduced for convenience of explanation. For example, because sizes and thicknesses of elements in the drawings are arbitrarily illustrated for convenience of explanation, the present disclosure is not necessarily limited thereto.

Hereinafter, embodiments of the present disclosure will be described in detail with reference to the accompanying drawings. When describing embodiments with reference to the accompanying drawings, the same or corresponding elements are denoted by the same reference numerals, and redundant descriptions thereof are omitted.

The present disclosure relates to a microphysiological system (MPS), an MPS manufacturing method, and an extracellular matrix coating method using the MPS.

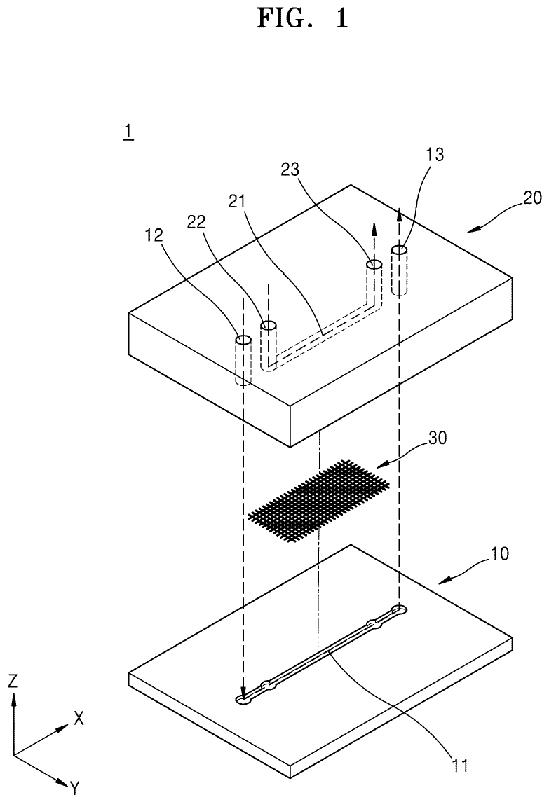

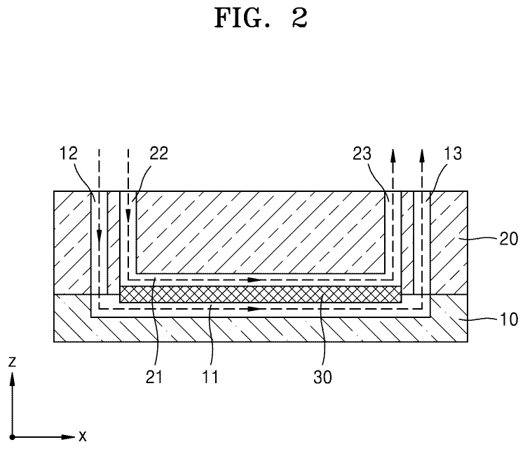

FIG. 1 is an exploded perspective view schematically illustrating an example of a microphysiological system (MPS) 1 according to an embodiment of the present disclosure, and FIG. 2 is a cross-sectional view schematically illustrating an example of the MPS 1 of FIG. 1.

Referring to FIGS. 1 and 2, the MPS 1 may include a substrate 10 having a first groove 11 formed on a surface thereof, a cover part 20 having a second groove 21 formed thereon and including a plurality of openings 12, 13, 22, and 23 formed in a thickness direction to cross the second groove 21, and a porous membrane 30 disposed between the substrate 10 and the cover part 20 and covering at least a portion of each of the first groove 11 and the second groove 21.

The substrate 10 may have the first groove 11 formed in a central portion of a surface thereof. For example, the first groove 11 may be formed with a length in a single direction in the central portion of the substrate 10 and may have a width in a direction crossing the length. Because the first groove 11 is formed with the length and the width, a space in which the fluid flows may be defined in the first groove 11. Therefore, the first groove 11 may serve as a microfluidic channel or chamber and may be connected to a first inlet 12 and a first outlet 13 described below to guide the flow of a first biofluid so that the first biofluid may circulate within the first groove 11.

In other embodiments, the first groove 11 may have an area where the width crossing the length is not constant and is larger. In this case, a plurality of areas having a larger width may be formed and may correspond to the first inlet 12 and the first outlet 13 described below.

The substrate 10 may be a transparent substrate, the interior of which may be observed from the outside. For example, the substrate 10 may be formed of any transparent material. As a specific example, the substrate 10 may include at least one selected from polydimethylsiloxane (PDMS), polyethersulfone (PES), poly(3,4-ethylenedioxythiophene), poly(styrenesulfonate), polyimide, polyurethane, polyester, perfluoropolyether (PFPE), and polycarbonate.

The cover part 20 may be disposed opposite the substrate 10. The cover part 20 may include the second groove 21. For example, the second groove 21 may be formed to have an area overlapping at least the first groove 11.

The cover part 20 may be a transparent substrate, the interior of which may be observed from the outside. For example, the cover part 20 may be formed of any transparent material. As a specific example, the cover part 20 may include at least one selected from PDMS, PES, poly(3,4-ethylenedioxythiophene), poly(styrenesulfonate), polyimide, polyurethane, polyester, PFPE, and polycarbonate.

As a specific example, the cover part 20 may have the second groove 21 formed on a surface of the cover part 20 facing the substrate 10.

In other embodiments, the second groove 21 may be formed in a central area or an area adjacent thereto on the surface of the cover part 20.

In some embodiments, the first groove 11 and the second groove 21 may overlap each other in at least one area to form a tubular shape. In this case, the second groove 21 may be formed to have a smaller length than the first groove 11 and may overlap the first groove 11 in an area of the first groove 11. For example, the second groove 21 may overlap the first groove 11 in a longitudinal direction on opposite sides along the center of the first groove 11, and opposite end portions of the second groove 21 may be located inside the opposite end portions of the first groove 11.

The plurality of openings 12, 13, 22, and 23 may be formed in a direction crossing the second groove 21, for example, a vertical thickness direction Z or in a direction perpendicular to the thickness direction Z, that is, a direction parallel to the second groove 21.

For example, the second groove 21 may face and overlap the first groove 11 to form a tubular shape and may be separated from the first groove 11 by the porous membrane 30 described below so that each of the first groove 11 and the second groove 21 may serve as a microfluidic channel or chamber. In this case, the depth of the microfluidic channel formed by the first groove 11 and the porous membrane 30 may be 150 μm to 250 μm, and the depth of the microfluidic channel formed by the second groove 21 and the porous membrane 30 may be 800 μm to 1,200 μm.

The plurality of openings 12, 13, 22, and 23 may include a first inlet 12, a first outlet 13, a second inlet 22, and a second outlet 23.

For example, the first inlet 12 and the first outlet 13 may be spaced apart from each other, and may be spaced apart from at least the second groove 21 and disposed outside the second groove 21.

In some embodiments, the first inlet 12 and the first outlet 13 may be formed to correspond to the first groove 11. For example, the first inlet 12 and the first outlet 13 may be formed to correspond to an end portion and another end portion of the first groove 11, respectively.

The first inlet 12 and the first outlet 13 may be formed to correspond to end portions of the first groove 11 formed in the substrate 10, respectively.

The second inlet 22 and the second outlet 23 may be spaced apart from each other, and may be spaced apart from at least the first inlet 12 and the first outlet 13 and disposed inside the first inlet 12 and the first outlet 13.

In some embodiments, the second inlet 22 and the second outlet 23 may be formed to correspond to the second groove 21 of the cover part 20. For example, the second inlet 22 and the second outlet 23 may be formed to correspond to an end portion and another end portion of the second groove 21, respectively.

In this case, the length of the first groove 11 may be equal to or different from the length of the second groove 21, and the first inlet 12 and the second inlet 22, and the first outlet 13 and the second outlet 23 may be spaced apart from each other.

The first inlet 12 and the first outlet 13 may be formed to overlap the first groove 11, for example, to correspond to opposite end portions of the first groove 11 so that the first inlet 12 and the first outlet 13 may be connected to the first groove 11 to form a flow path through which the fluid flows. In this case, the first inlet 12 and the first outlet 13 may be attached to or detached from a tube connected to an external pump, and thus, the flow of the fluid circulating through the first groove 11 may be controlled at a desired speed through the external pump.

The second inlet 22 and the second outlet 23 may be formed to overlap the second groove 21, for example, to correspond to opposite end portions of the second groove 21 so that the second inlet 22 and the second outlet 23 may be connected to the second groove 21 to form a flow path through which the fluid flows. In this case, the second inlet 22 and the second outlet 23 may be attached to or detached from the tube connected to the external pump, and thus, the flow of the fluid circulating through the second groove 21 may be controlled at a desired speed through the pump.

For example, the first inlet 12, the first outlet 13, the second inlet 22, and the second outlet 23 may all be attached to or detached from the tube, and the tube may be connected to the external pump so that the flow of the fluid injected into the first groove 11 and the second groove 21 and then discharged by the pump may be controlled.

In other embodiments, the first groove 11 may include areas having different widths. For example, in FIG. 1, the first groove 11 may include four areas having different widths. As a specific example, the first groove 11 may have four areas which are convex on opposite sides to have large widths (hereinafter, defined as “convex areas”). The four convex areas of the first groove 11 may correspond to and overlap the plurality of openings 12, 13, 22, and 23, respectively.

In some embodiments, the first groove 11 and the plurality of openings 12, 13, 22, and 23 may be formed perpendicular to each other. Due to coupling parts formed to be perpendicular with right angles, the fluid introduced into the plurality of openings 12, 13, 22, and 23, the first groove 11, and the second groove 21 may experience rapid changes in flow velocity and pressure. In this case, because the convex areas overlapping the plurality of openings 12, 13, 22, and 23 are formed in the first groove 11, spaces may be formed in the convex areas. The coupling parts of the first groove 11 and the plurality of openings 12, 13, 22, and 23, which are vertically connected at right angles, may have a streamlined shape, thereby preventing the flow direction of the fluid from changing abruptly. Therefore, the flow velocity and hydraulic pressure of the fluid flowing through the first groove 11 and the second groove 21 may be prevented from changing, and shear stress caused by the fluid flowing through the first groove 11 and the second groove 21 may be maintained constant.

The fluid introduced into the first groove 11 and the second groove 21 may include a biofluid. For example, the biofluid may circulate within the first groove 11 by being injected into the first groove 11 through the first inlet 12 and discharged to the exterior through the first outlet 13, and may circulate within the second groove 21 by being injected into the second groove 21 through the second inlet 22 and discharged to the exterior through the second outlet 23.

In other embodiments, the biofluid may be injected into the first groove 11 through the first inlet 12 and remain in a static state within the first groove 11 without being discharged, and may circulate within the second groove 21 by being injected into the second groove 21 through the second inlet 22 and discharged to the exterior through the second outlet 23.

In other embodiments, the biofluid may circulate within the first groove 11 by being injected into the first groove 11 through the first inlet 12 and discharged to the exterior through the first outlet 13, and may be injected into the second groove 21 through the second inlet 22 and remain in a static state within the second groove 21 without being discharged.

In other embodiments, the biofluid may be injected into the first groove 11 through the first inlet 12 and remain in a static state within the first groove 11 without being discharged, and may be injected into the second groove 21 through the second inlet 22 and remain in a static state within the second groove 21 without being discharged.

The biofluid may include, for example, a material selected from circulating cells, adherent cells, scaffolds, chemicals, and biomolecules. The circulating cells and the adherent cells may include any types of cells, such as endothelial cells, epithelial cells, stromal cells, or immune cells derived from animals including humans. The scaffolds may include proteins or hydrogels including constituent molecules of cells including extracellular matrix. The chemicals may include cell culture media, and additives and drugs added to the cell culture media. The biomolecules may include any molecules produced by organisms, such as signaling molecules or neurotransmitters. However, the present disclosure is not limited thereto. The biomolecules may include any types of substances which are generally available in the past, depending on the experimental purpose.

The porous membrane 30 may be disposed between the substrate 10 and the cover part 20. For example, the porous membrane 30 may be disposed between the first groove 11 and the second groove 21.

As a specific example, the porous membrane 30 may be disposed to overlap the first groove 11 and the second groove 21 and may be disposed to cover at least one area of the space between the first groove 11 and the second groove 21.

For example, the porous membrane 30 may include a polyethylene terephthalate (PET) porous membrane, but the present disclosure is not limited thereto, and the porous membrane 30 may include other materials. The porous membrane 30 may include any material which is usable as the porous membrane 30.

The porous membrane 30 may be disposed between the first groove 11 and the second groove 21 in an area where the first groove 11 and the second groove 21 overlap each other, so that the porous membrane 30 may selectively migrate, to the second groove 21, the biofluid injected into the first groove 11, or may selectively migrate, to the first groove 11, the biofluid injected into the second groove 21.

In other embodiments, the porous membrane 30 may selectively migrate, to the first groove 11, the biofluid including circulating tumor cells injected into the second groove 21. In this case, endothelial cells cultured on the surface of the porous membrane 30 facing the second groove 21 may be infiltrated by the circulating tumor cells injected into and migrated along the second groove 21.

In the case of the cancer cells, co-culturing with the endothelial cells may weaken the intercellular bonds of the endothelial cells to induce condensation of the endothelial cells. Due to this, the intercellular bonds of the endothelial cells are weakened, and thus, the collapse of the endothelial cell barrier cultured on the porous membrane 30 may easily occur. The collapse of the endothelial cell barrier may cause disordered transendothelial migration (TEM) of the cancer cells through the endothelial cells, making it difficult to maintain the membrane permeability of the porous membrane 30, on which the endothelial cells are cultured, similar to an in vivo environment.

To solve this problem, in the present disclosure, an extracellular matrix oriented in a single direction may be coated on the porous membrane 30. For example, to coat the extracellular matrix oriented in the single direction, a constant flow may be applied to the biofluid including the extracellular matrix. In case that such a constant flow is applied, the extracellular matrix may be oriented and aligned in the single direction and a fibrous extracellular matrix coating layer may be formed.

In other embodiments, in case that endothelial cells and cancer cells are co-cultured in the second groove 21, the biofluid including the extracellular matrix may be first introduced into the second groove 21, and thus, the extracellular matrix may be coated on the second groove 21 and the surface of the porous membrane 30 facing a second groove direction. In this case, because a constant flow may be applied to the biofluid, the extracellular matrix may be oriented and aligned in the single direction and the fibrous extracellular matrix coating layer may be formed on the second groove 21 and the surface of the porous membrane 30 facing the second groove direction.

As described above, endothelial cells may be introduced and oriented on the fibrous extracellular matrix coating layer in which the extracellular matrix is oriented and aligned.

Endothelial cells cultured in the aligned fibrous structures may exhibit enhanced cell proliferations and increased gene expression associated with intercellular adhesion, thereby enabling excellent barrier functions to be provided without collapsing even when co-cultured with cancer cells.

As a result, the barriers of the endothelial cells cultured on the aligned fibrous extracellular matrix coating layer may maintain structural stability even in an environment co-cultured with the cancer cells, which may provide a physiological advantage of restricting disordered TEM of the cancer cells and maintaining membrane permeability.

The first groove 11 and the second groove 21 of the MPS 1 may be separated from each other by the porous membrane 30 to form a linear microfluidic channel or chamber, that is, to form a linear microfluidic channel or chamber including an inlet and an outlet separately formed in each of the first groove 11 and the second groove 21. Therefore, various treatments such as cells, scaffolds, or drugs may be performed in the microfluidic channel of each of the first groove 11 and the second groove 21 according to a model to be applied.

In other embodiments, in case that a circulation model is applied to the MPS 1, the extracellular matrix may be injected into the second groove 21 through the second inlet 22 of the MPS 1. A poly-L-lysine (PLL) coating agent and an extracellular matrix protein, which are injected through the second inlet 22 and are easy for cell adhesion, may be discharged through the second outlet 23 to coat the second groove 21. After the second groove 21 is coated, human pulmonary vascular endothelial cells and a culture medium may be injected and discharged again through the second inlet 22 and the second outlet 23 to form a monolayer of human pulmonary vascular endothelial cells on the surface of the second groove 21. Accordingly, in vivo blood vessels may be simulated in the second groove 21.

In case that the blood vessels are simulated in the second groove 21, cancer cells may circulate in the second groove 21 through the second inlet 22 and the second outlet 23, and cancer cells (circulating tumor cells) circulating in the second groove 21 may be attached onto the simulated endothelial cells. In this case, some cancer cells may be migrated through the porous membrane 30 to the first groove 11 and infiltrated into the extracellular matrix formed in the first groove 11. In this case, to facilitate the distinction between the endothelial cells and the cancer cells, CellTracker™ may be used to label the pulmonary vascular endothelial cells with green 5-chloromethylfluorescein diacetate (CMFDA) dye and to label the circulating tumor cells with red 5-(and-6)-carboxyfluorescein diacetate succinimidyl ester (CMFPX) dye.

Because each of the first groove 11 and the second groove 21 has a linear structure, in case that a first biofluid and a second biofluid are injected into and circulated in the microfluid channel while adjusting the flow rates of the first biofluid and the second biofluid, the extracellular matrix and the coating agent may be uniformly coated throughout the microfluidic channel or chamber, the human pulmonary vascular cells may be uniformly cultured as a monolayer, and circulating tumor cells may be uniformly attached to the endothelial cells.

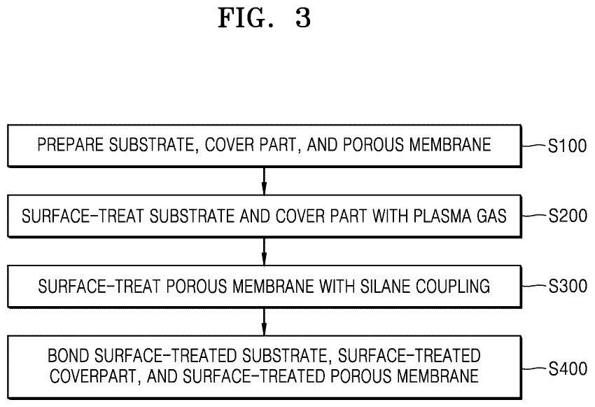

FIG. 3 is a flowchart illustrating an example of an MPS manufacturing method according to another embodiment of the present disclosure.

Referring to FIG. 3, the MPS manufacturing method may include preparing a substrate, a cover part, and a porous membrane (S100), surface-treating the substrate and the cover part with plasma gas (S200), surface-treating the porous membrane with a silane coupling agent (S300), and bonding the surface-treated substrate, the surface-treated cover part, and the surface-treated porous membrane (S400).

In the preparing of the substrate, the cover part, and the porous membrane (S100), the substrate 10 and the cover part 20 may be manufactured by using a mold including the shapes of the first groove 11 and the second groove 21 by milling with a computerized numerical control (CNC) machine. The substrate 10 and the cover part 20 may be prepared by a molding method in which liquid PDMS is poured into the mold to imitate the shape of the mold, and the cover part 20 may be prepared by additionally forming a plurality of openings with a desired diameter by using a puncher in the PDMS imitated in the mold.

The porous membrane may be prepared by manufacturing a PET thin-film to correspond to the length of the second groove 21.

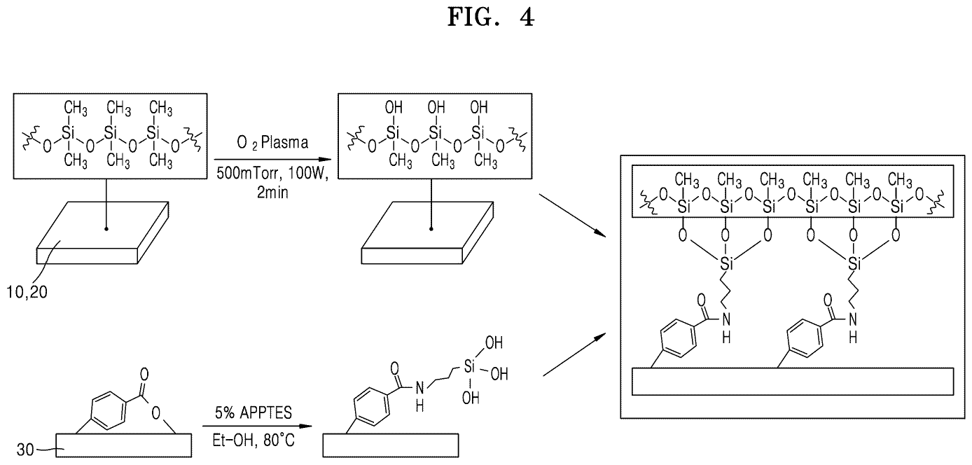

FIG. 4 is a process diagram schematically illustrating an example of the MPS manufacturing method of FIG. 3.

Referring to FIG. 4, in the surface-treating of the substrate and the cover part with plasma gas (S200), the substrate 10 and the cover part 20 may be surface-treated with oxygen plasma (O₂ plasma). For example, the PDMS which forms the substrate 10 and the cover part 20 has a surface composed of CH₃⁻ and thus has hydrophobicity. However, in case that the surface of the PDMS is treated with oxygen plasma (O₂ plasma), the surface of the PDMS may be substituted from CH₃⁻ to OH⁻ and thus become hydrophilic. In this case, the treatment may be performed with the oxygen plasma (O₂ plasma) for 1 minute to 3 minutes under conditions of 400 m Torr to 500 m Torr and 80 W to 120 W.

In the surface-treating of the porous membrane with the silane coupling agent (S300), the porous membrane 30 may be surface-treated with the silane coupling agent, and the silane coupling agent may include a (3-aminopropyl)triethoxysilane (APTES) coupling agent. In this case, the APTES coupling agent may be prepared by adding 5% mol of APTES to a solvent including ethanol (Et-OH) or distilled water (DW), and may be heated to 70° C. to 90° C. and treated on the surface of the porous membrane 30.

The porous membrane 30 surface-treated with the APTES may have a surface modified with an amino group.

In the bonding of the surface-treated substrate, the surface-treated cover part, and the surface-treated porous membrane (S400), the substrate 10 and the cover part 20 which have been surface-treated with the oxygen plasma (O₂ plasma) may have surfaces which exhibit a hydroxyl group (OH⁻), and the porous membrane 30 which has been surface-treated with the APTES coupling agent may have a surface which exhibits an amino group. Accordingly, the substrate 10 and the cover part 20 may form a strong covalent bond with the porous membrane 30. Therefore, the substrate 10, the cover part 20, and the porous membrane 30 may be firmly attached to each other by disposing the porous membrane 30 between the substrate 10 and the cover part 20.

The bonding strength between the substrate 10, the cover part 20, and the porous membrane 30 may vary depending on the solvent used in the APTES coupling agent.

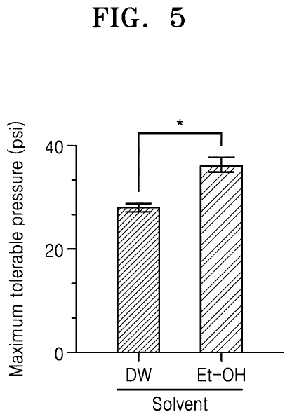

FIG. 5 is a graph showing a bonding strength of a silane coupling agent according to a solvent.

Referring to FIG. 5, in case that DW is used as a solvent, the fluid pressure which the MPS may withstand without fluid leakage may be about 23 psi, and in case that Et-OH is used as a solvent, the fluid pressure which the MPS may withstand without fluid leakage may be about 35 psi. Accordingly, it may be confirmed that the bonding strength is about 1.5 times stronger in case that Et-OH is used as a solvent than in case that DW is used as a solvent.

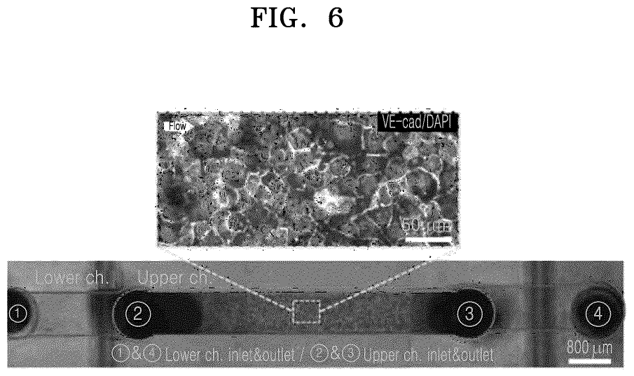

FIG. 6 is an image showing a cell culture result of an MPS manufactured by the MPS manufacturing method of FIG. 3;

Referring to FIG. 6, human pulmonary vascular endothelial cells cultured in a microfluidic channel were stained with VE-cadherin (endothelial specific adhesion molecule, white) and DAPI (nuclear DNA, blue) markers, and then, fluorescence analysis was performed to confirm that the human pulmonary vascular endothelial cells injected into the microfluidic channel formed a monolayer on the surface of the microfluidic channel, and that in vivo blood vessels were simulated in the microfluidic channel.

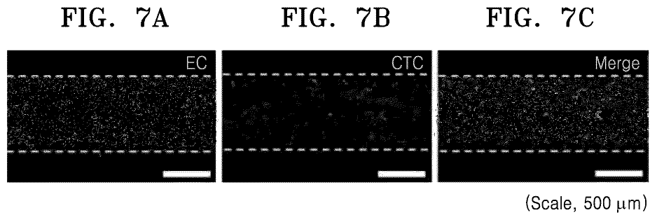

FIGS. 7A, 7B, 7C are images showing a test result of the MPS manufactured by the MPS manufacturing method of FIG. 3;

Referring to FIGS. 7A, 7B, 7C, FIG. 7A shows human pulmonary vascular endothelial cells cultured in the second groove, FIG. 7B shows circulating tumor cells circulating in the second groove, and FIG. 7C shows cancer cells attached to human pulmonary vascular endothelial cells formed in the second groove.

Referring to FIGS. 7A, 7B, 7C, as a result of culturing pulmonary vascular endothelial cells by using the MPS of the present disclosure, it may be confirmed that pulmonary vascular endothelial cells are cultured as a monolayer at a uniform density in a microfluidic channel having a linear structure, and it may be confirmed that cancer cells are uniformly attached to the top of the endothelial cells without fluid leakage even during the circulation of cancer cells using tubing.

As a result, the MPS may maintain a constant circulation velocity of fluid within the microfluidic channel to induce uniform-density adhesion of cells on the surface of the microfluidic channel during cell culture and circulation, and may be bonded by using the oxygen plasma and the silane coupling agent and thus attached immediately and strongly through chemical bonding.

Hereinafter, an extracellular matrix coating method using an MPS, according to an embodiment of the present disclosure, is described.

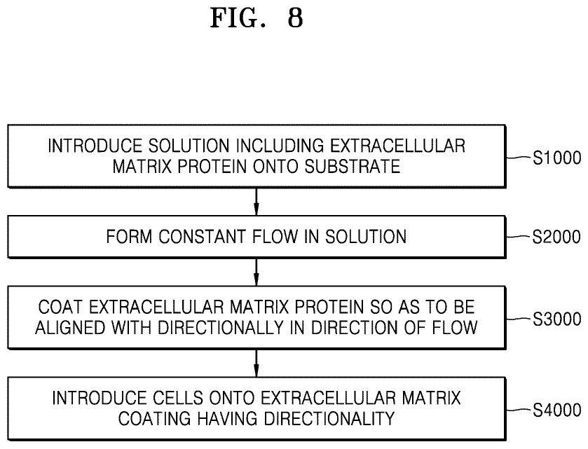

FIG. 8 is a flowchart illustrating an example of an extracellular matrix coating method according to another embodiment of the present disclosure.

Referring to FIG. 8, the extracellular matrix coating method may include introducing a solution including an extracellular matrix protein onto a substrate (S1000), forming a constant flow in the solution (S2000), coating the extracellular matrix protein so as to be aligned directionally in the direction of the flow (S3000), and introducing cells onto the extracellular matrix coating having the directionality (S4000).

In the introducing of the solution including the extracellular matrix protein onto the substrate (S1000), the substrate may be a transparent substrate, the interior of which may be observed from the outside. For example, the substrate may include silicon. As a specific example, the substrate may include PDMS. As a more specific example, the substrate may include the MPS according to an embodiment of the present disclosure.

In other embodiments, the substrate may include a first groove and a second groove which is disposed to overlap the first groove. In this case, a porous membrane may be disposed between the first groove and the second groove. Accordingly, a space may be defined by the first groove and a surface of the porous membrane facing a first groove direction, and a space may be defined by the second groove and another surface of the porous membrane facing a second groove direction.

The solution including the extracellular matrix may be injected into the space defined by the second groove and the other surface of the porous membrane.

In the forming of the constant flow in the solution (S2000), a pump located outside the substrate may apply constant shear stress to the solution injected into the space defined by the second groove and the other surface of the porous membrane, and thus, the constant flow may be formed in the solution.

In the coating of the extracellular matrix protein so as to be aligned directionally in the direction of the flow (S3000), the extracellular matrix included in the solution in which the constant flow is formed in the space defined by the second groove and the other side of the porous membrane may be oriented and aligned in a single direction in the space defined by the second groove and the other surface of the porous membrane.

By sequentially injecting blood plasma and calcium chloride (CaCl₂) with applied flow onto the extracellular matrix oriented as described above, the extracellular matrix included in the solution may be coated in a fibrous form on the second groove and the other surface of the porous membrane.

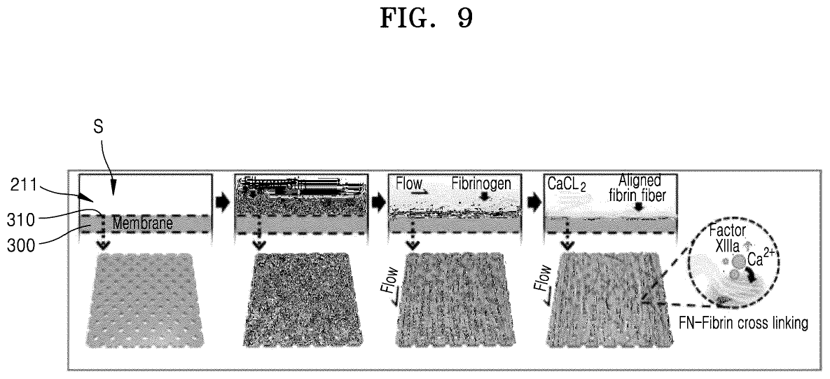

FIG. 9 is a diagram schematically illustrating an example of the extracellular matrix coating method of FIG. 8.

Referring to FIG. 9, an extracellular matrix protein may be aligned and coated in a single direction on another surface 310 of a porous membrane 300.

A solution having a constant flow in a space S defined by a second groove 211 and the other surface 310 of the porous membrane 300 may include, for example, fibronectin, which is an extracellular matrix. In case that the solution including the fibronectin is injected with a constant flow into the space S defined by the second groove 211 and the other surface 310 of the porous membrane 300, the fibronectin may be disposed on the other surface 310 of the porous membrane 300.

In case that the fibronectin is disposed on the other surface 310 of the porous membrane 300, blood plasma including fibrinogen may be injected with a constant flow into the space S defined by the second groove 211 and the other surface 310 of the porous membrane 300, and thus, the fibrinogen may be disposed on the fibronectin.

Next, in case that calcium chloride (CaCl₂) is injected with a constant flow onto the fibronectin and the fibrinogen, factor XIIIa is activated, causing the fibronectin and fibrin to be cross-linked and aligned so that the extracellular matrix may be coated on the other surface 310 of the porous membrane 300 in the form of fibrin fibers.

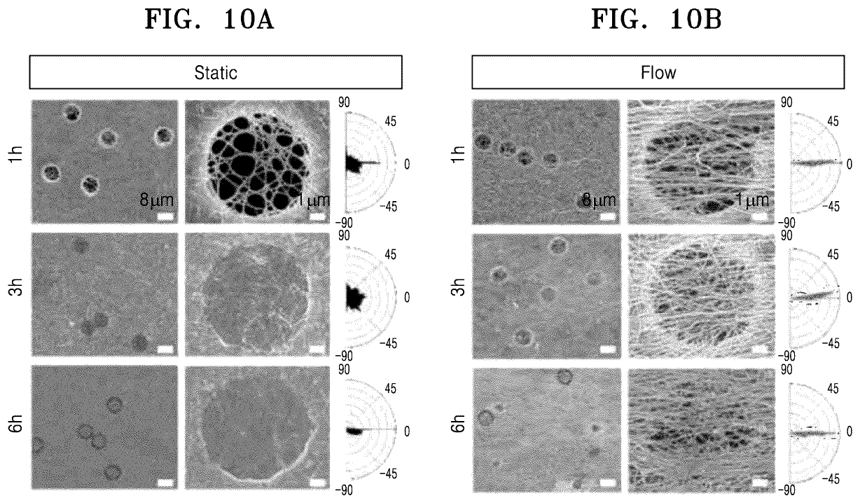

FIGS. 10A and 10b are images showing the alignment of the extracellular matrix coated by the extracellular matrix coating method of FIG. 8.

Referring to FIGS. 10A and 10B, the orientation of the extracellular matrix under a static condition (a) and a flow condition (b) may be confirmed.

As a result of performing plasma treatment on the other surface 310 of the porous membrane 300 at intervals of 1 hour, 3 hours, and 6 hours, random orientation was observed in the static condition (a), but the fibrin fibers aligned along the direction of fluid flow was observed in the flow condition (b).

In the introducing of the cells onto the extracellular matrix coating having the directionality (S4000), cells may be cultured on the extracellular matrix coating having the directionality. In this case, the cells may be oriented and cultured along the extracellular matrix having the directionality.

The cells are not limited to a single type of cell, but various types of cells may be introduced and cultured on the extracellular matrix depending on the experimental purpose.

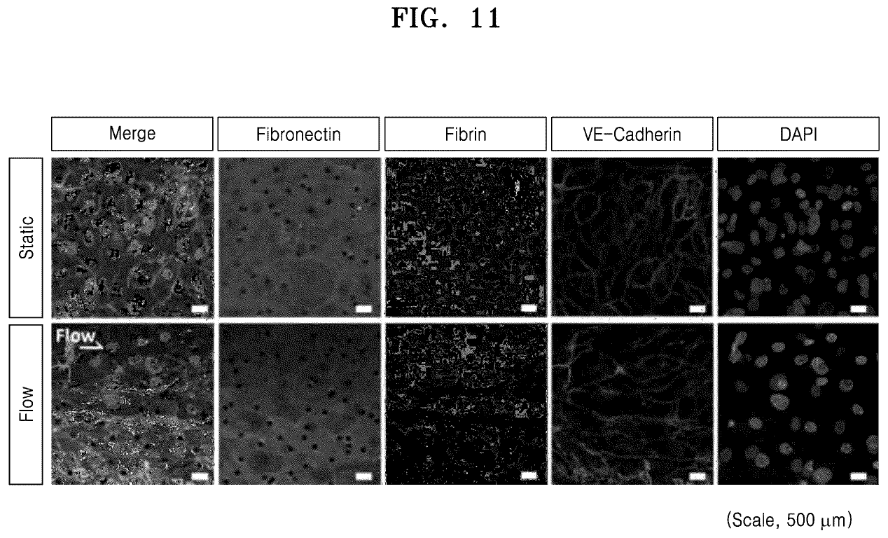

FIG. 11 is a fluorescence analysis image of cells cultured on an extracellular matrix coated by the extracellular matrix coating method of FIG. 8.

Referring to FIG. 11, human umbilical vein endothelial cells (HUVECs), which were stainable with fibronectin (gray), fibrin (green), VE-cadherin (red), and DAPI (blue) markers and were cultured under a static condition and a flow condition, were confirmed.

As a result of culturing HUVECs on a substrate coated with fibrin fibers, it was confirmed that HUVECs cultured on the coated substrate under the static condition showed irregular cell arrangement, whereas HUVECs cultured along the coated aligned fibers under the flow condition were arranged with directionality.

### Example 1

After flowing a solution including fibronectin with a constant flow through a microfluidic channel of an MPS, blood plasma and calcium chloride with a constant flow were sequentially flowed so that HUVECs were cultured on a membrane coated with fibrin fibers aligned with an orientation in a flow direction.

### Example 2

By statically locating a solution including fibronectin, blood plasma, and calcium chloride in a microfluidic channel of an MPS, HUVECs were cultured on a membrane coated with fibrin fibers.

### Comparative Example 1

By flowing a solution including fibronectin through a microfluidic channel of an MPS, HUVECs were cultured on a membrane coated with fibronectin.

### Comparative Example 2

HUVECs were cultured on an experimental well plate coated with fibronectin.

### Comparative Example 3

HUVECs were cultured on an uncoated experimental well plate.

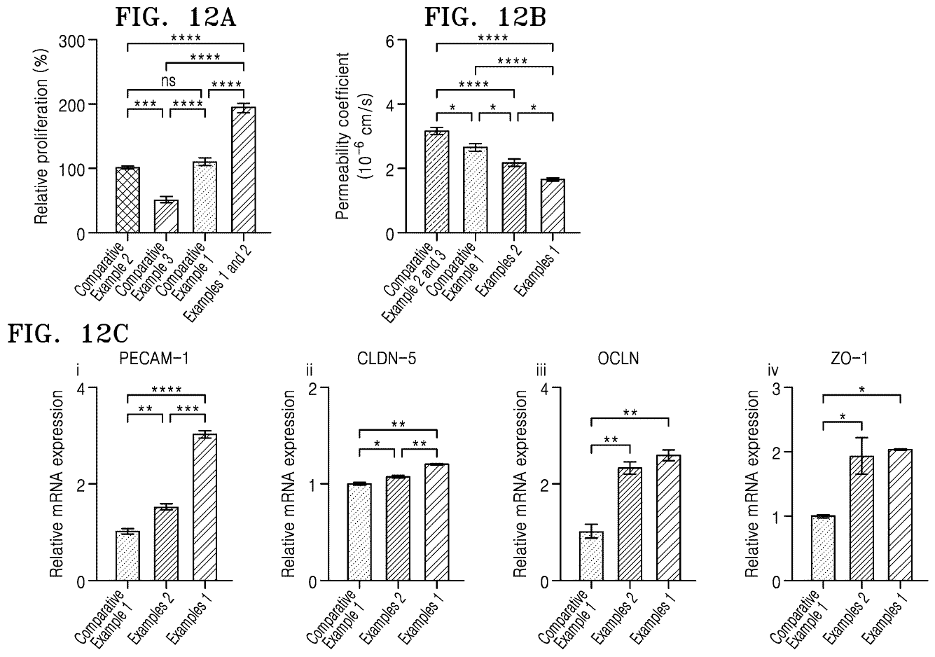

FIGS. 12A, 12B, 12C are graphs evaluating the functions of cells cultured on extracellular matrices coated under various conditions;

Referring to FIGS. 12A, 12B, 12C, FIG. 12A of cell proliferation, FIG. 12B of permeability, and FIG. 12C of endothelial cell junction marker (PECAM-1) and tight junction marker (CLDN-5, OCLN, ZO-1) analysis in Examples 1 and 2 and Comparative Examples 1 to 3 may be confirmed.

Referring to graph (a) and graph (b), it may be confirmed that HUVECs cultured in Example 1 exhibited an excellent barrier function due to high proliferation and lowest permeability, compared to Example 2 and Comparative Examples 1 to 3.

Referring to graph (c), it may be confirmed through analysis of gene expression associated with intercellular adhesion and attachment that the functions of HUVECs cultured in Example 1 were improved.

### Experimental Example 1

MDA-MB-231 serving as a highly metastatic breast cancer cell line was co-cultured with HUVECs cultured in Example 1.

### Experimental Example 2

MDA-MB-231 serving as a highly metastatic breast cancer cell line was co-cultured with HUVECs cultured in Example 2.

### Experimental Example 3

MDA-MB-231 serving as a highly metastatic breast cancer cell line was co-cultured with HUVECs cultured in Comparative Example 1.

### Experimental Example 4

MCF-7 serving as a low metastatic breast cancer cell line was co-cultured with HUVECs cultured in Example 1.

### Experimental Example 5

MCF-7 serving as a low metastatic breast cancer cell line was co-cultured with HUVECs cultured in Example 2.

### Experimental Example 6

MCF-7 serving as a low metastatic breast cancer cell line was co-cultured with HUVECs cultured in Comparative Example 1.

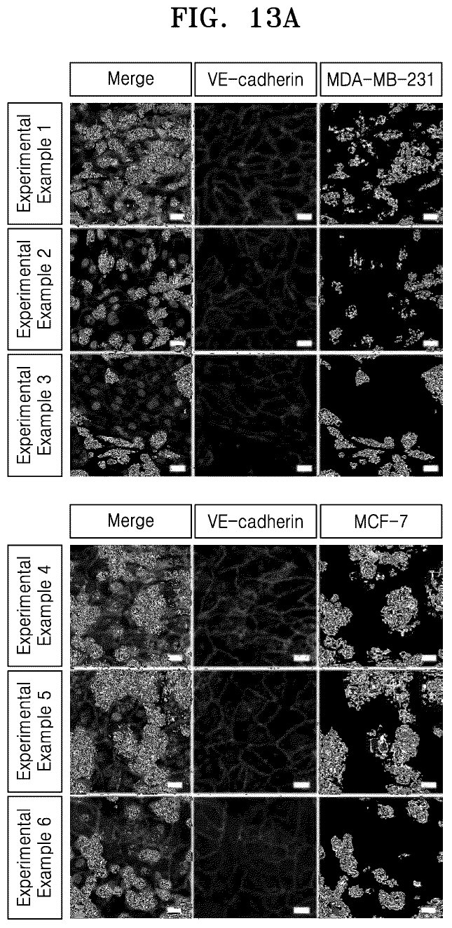

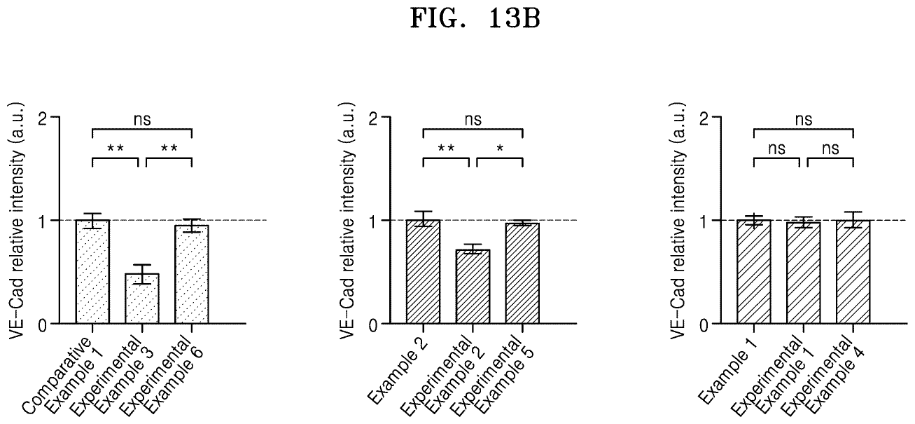

FIG. 13A is a fluorescence analysis image of a co-culture of cells cultured on extracellular matrices under various conditions and a cancer cell line, and FIG. 13B is a graph showing relative quantification of fluorescence-labeled tight junction proteins in FIG. 13A.

Referring to FIGS. 13A and 13B, the barrier functions of endothelial cell layers may be evaluated by using two breast cancer cell lines having different metastatic properties.

Experimental Examples 1 to 3 are co-cultures of highly metastatic MDA-MB-231, which has the property of migrating while destroying the tight junctions of endothelial cells, with HUVECs of Examples 1 and 2 and Comparative Example 1, and Experimental Examples 4 to 6 are co-cultures of low metastatic MCF-7, which has a superior binding strength between cancer cells, with HUVECs of Examples 1 and 2 and Comparative Example 1.

Intercellular adhesion of endothelial cells was fluorescence-quantified by using tight junction protein (VE-Cadherin) as a marker.

According to the analysis result, it was confirmed that the HUVECs cultured in Experimental Example 1 maintained barriers because the HUVECs maintained the same quantification of tight junction protein (VE-Cadherin) as in Example 1, which was not co-cultured with highly metastatic MDA-MB-231. In contrast, it was confirmed that the HUVECs cultured in Experimental Examples 2 and 3 showed barrier collapse because the quantity of tight junction protein (VE-cadherin) was reduced, compared to Example 2 and Comparative Example 1, which were not co-cultured with MDA-MB-231.

Furthermore, it was confirmed that Experimental Examples 4 to 6 maintained barriers because Experimental Examples 4 to 6 maintained the same quantification of tight junction protein as Examples 1 and 2 and Comparative Example 3, which were not co-cultured with low metastatic MCF-7.

In other words, it was confirmed that the HUVECs coated on the extracellular matrix aligned with directionality maintained barriers without collapsing when co-cultured with highly metastatic MDA-MB-231 or low metastatic MCF-7.

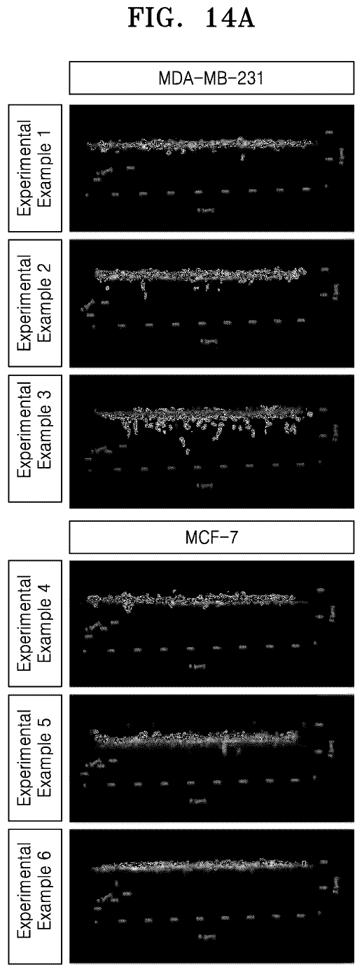

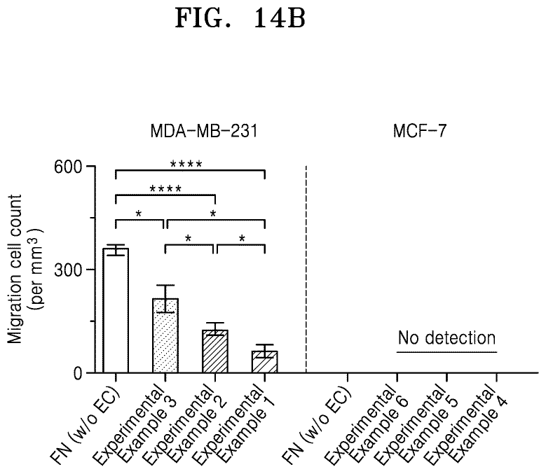

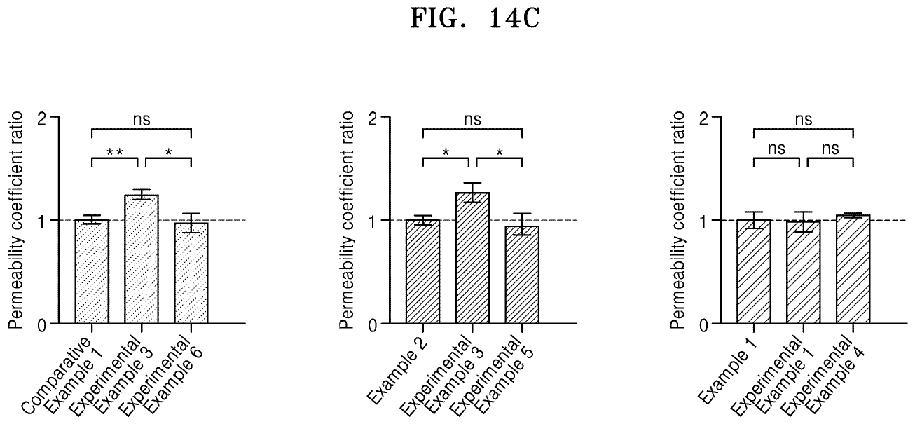

FIG. 14A is a fluorescence analysis image of metastasis of cancer cells when cells cultured on extracellular matrices under various conditions and a cancer cell line were co-cultured, FIG. 14B is a graph showing the number of fluorescence-labeled cancer cells per volume in FIG. 14A, and FIG. 14C is a graph showing the permeability coefficient due to metastasis of cancer cells when cells cultured on extracellular matrices under various conditions and a cancer cell line were co-cultured.

Referring to FIG. 14A, to facilitate cell distinction, HUVECs may be stained with red CMFDA dye and cancer cells may be stained with green CMFPX dye. It was confirmed that, when co-cultured with highly metastatic MDA-MB-231, fewer metastatic cancer cells (green marker) were observed in Experimental Example 1, compared to Experimental Examples 2 and 3.

Furthermore, it was confirmed that, when co-cultured with low metastatic MCF-7, metastatic cancer cells (green marker) were hardly observed in Experimental Examples 4 to 6.

Referring to FIG. 14B, MDA-MB-231 having strong tight junction destruction characteristics showed metastatic behavior in all of Experimental Examples 1 to 3, but it was confirmed that the number of metastatic cancer cells confirmed in Experimental Example 1 was the lowest, and thus, it was seen that the vascular endothelial cells of Experimental Example 1 showed the lowest cancer cell infiltration, compared to Experimental Examples 2 and 3. In other words, it was seen that the barrier functions of the vascular endothelial cells of Experimental Example 1 were improved, compared to Experimental Examples 2 and 3.

Such results may also be confirmed by comparing the permeability coefficients of the vascular endothelial cells.

Referring to FIG. 14C, Experimental Example 1 maintains 1, while Experimental Examples 2 and 3 exceed 1. Therefore, it may be seen that the barrier functions of the vascular endothelial cells of Experimental Example 1 were improved, compared to Experimental Examples 2 and 3.

In other words, it was confirmed that the HUVECs coated on the extracellular matrix aligned with directionality maintained barriers without collapsing when co-cultured with highly metastatic MDA-MB-231 or low metastatic MCF-7.

As a result, the extracellular matrix coating method according to an embodiment of the present disclosure may implement a robust cell barrier similar to an in vivo environment by coating an extracellular matrix aligned with directionality on an MPS in a fibrous form.

Furthermore, the extracellular matrix coating method according to an embodiment may be effectively used to develop an MPS and conduct experimental research using the same by complementing the physiological environment of the in vivo basement membrane in vitro and providing various biological functions, such as enhancing proliferation and gene expression of endothelial cells, regulating permeability, and maintaining endothelial cell barriers in cancer cell co-culture environments.

The present disclosure has been described with reference to one or more embodiments illustrated in the drawings, but this is only an example. It will be understood by those of ordinary skill in the art that various modifications and equivalents may be made thereto. Therefore, the true technical protection scope of the present disclosure should be defined by the technical concept of the appended claims.

Specific executions described in the embodiments are one embodiment, which does not limit the scope of the embodiments in any way. For the sake of conciseness of the specification, descriptions of conventional electronic components, control providing methods, software, and other functional aspects of the control providing methods may be omitted. In addition, connecting lines or connecting members illustrated in the drawings are intended to represent example functional connections and/or physical or circuit connections. In an actual device, it may appear as a variety of alternative or additional functional, physical, or circuit connections. Furthermore, when there is no specific mention such as “essential,” “important,” etc., it may not be a necessary component for the application of the present disclosure.

The use of the term “the” and similar demonstratives in the specification of the embodiments (in particular, the claims) is to be construed to cover both the singular and the plural. Furthermore, when a range is described in the embodiments, it includes the invention to which individual values within the range are applied (unless otherwise indicated herein). This is the same as stating each individual value constituting the above range in the detailed description. Finally, operations constituting methods according to embodiments may be performed in any suitable order unless otherwise indicated herein or otherwise clearly contradicted by context. The embodiments are not necessarily limited by the order of description of operations. The use of any and all examples or exemplary terms (e.g., “such as”) provided in the embodiments is simply intended to describe the embodiments in detail, and the scope of the embodiments is not limited by the examples or exemplary terms unless otherwise claimed. In addition, it will be understood by those of ordinary skill in the art that various modifications, combinations and changes may be made according to design conditions and factors within the scope of the appended claims or equivalents thereof.

1. A microphysiological system comprising:
a substrate having a first groove formed on a surface thereof;
a cover part disposed opposite the substrate and having a second groove formed on a surface thereof facing the substrate;
a porous membrane disposed between the substrate and the cover part so as to have an area overlapping at least the first groove and the second groove; and
a plurality of openings having a first inlet, a first outlet, a second inlet, and a second outlet, which pass through the cover part in a thickness direction or in a direction perpendicular to the thickness direction.

2. The microphysiological system of claim 1, wherein a length of the first groove is greater than or equal to a length of the second groove,
the first inlet and the first outlet correspond to end portions of the first groove, respectively, and
the second inlet and the second outlet correspond to end portions of the second groove, respectively.

3. The microphysiological system of claim 1, wherein the first groove has a length extending in a single direction and a width crossing the length, and the width comprises a plurality of convex areas spaced apart from each other with a larger width along the length.

4. The microphysiological system of claim 3, wherein the plurality of convex areas overlap the first inlet, the first outlet, the second inlet, and the second outlet.

5. The microphysiological system of claim 1, wherein a biofluid is injected into the first groove and the second groove.

6. The microphysiological system of claim 5, wherein the biofluid comprises a material selected from circulating cells, adherent cells, scaffolds, chemicals, and biomolecules.

7. The microphysiological system of claim 5, wherein the biofluid is selectively exchanged through the porous membrane in an area where the first groove and the second groove overlap each other.

8. The microphysiological system of claim 6, wherein the circulating cells and the adherent cells are cultured in a single-layer or multi-layer structure or are cultured while circulating along channels of the microphysiological system.

9. The microphysiological system of claim 6, wherein the scaffolds comprise an extracellular matrix protein or a hydrogel protein.

10. The microphysiological system of claim 6, wherein the chemicals comprise cell culture media and additives.

11. The microphysiological system of claim 6, wherein the biomolecules comprise signaling molecules or neurotransmitters.

12. A method of manufacturing a microphysiological system, the method comprising:
preparing a substrate, a cover part, and a porous membrane;
surface-treating the substrate and the cover part with plasma gas; surface-treating the porous membrane with a silane coupling agent; and
bonding the surface-treated substrate, the surface-treated cover part, and the surface-treated porous membrane.

13. The method of claim 12, wherein the plasma gas comprises oxygen or air, and the oxygen or the air modifies surfaces of the substrate and the cover part with a hydroxyl group.

14. The method of claim 12, wherein the silane coupling agent comprises water or ethanol as a solvent.

15. An extracellular matrix coating method comprising:
introducing a solution including an extracellular matrix protein onto a substrate;
forming a constant flow in the solution;
coating the extracellular matrix protein so as to be aligned directionally in a direction of the flow; and
introducing cells onto the extracellular matrix coating having the directionality.

16. The extracellular matrix coating method of claim 15, wherein blood plasma and calcium chloride (CaCl₂) are further injected during the coating while forming the constant flow.
